(19) United States
(12) Patent Application Publication
(10) Pub. No.: US 2007/0149870 A1
(43) Pub. Date: Jun. 28, 2007

(54) Systems and methods for determining an organism's pathology

(75) Inventor: Robert D. Rosenthal, Boca Raton, FL (US)

Correspondence Address: ROTHWELL, FIGG, ERNST & MANBECK, P.C., 1425 K STREET, N.W., SUITE 800, WASHINGTON, DC, 20005, US

(73) Assignee: FUTREX, Inc., Gaithersburg, MD (US)

(21) Appl. No.: 11/646,302

(22) Filed: Dec. 28, 2006

Related U.S. Application Data
(60) Provisional application No. 60/754,197, filed on Dec. 28, 2005.

(51) Int. Cl. A61B 5/00; A61B 5/02

(52) U.S. Cl. 600/323; 600/324; 600/500

(57) ABSTRACT

A system and method for detecting whether a subject has a physiological abnormality. The system includes a fingertip sensor and a data collection and analysis system coupled to the fingertip sensor.

This application claims the benefit of U.S. Provisional Patent Application No. 60/754,197, filed on Dec. 28, 2005, which application is incorporated herein by this reference.

### BACKGROUND

1. Field of the Invention

The present invention relates to systems and methods for detecting whether an organism has physiological abnormalities.

2. Discussion of the Background

During the last eighty years, thanks to advances in medical science, deaths from infectious diseases such as pneumonia, measles, diphtheria, and many others, have sharply diminished. The result of these advances is that the average person lives considerably longer and healthier lives than they used to.

One byproduct of the longer life span has become the increasingly important need to prevent or delay the onset of the adult diseases such as cancer, strokes and heart attacks. These adult diseases are now the primary cause of death in the developed world. Because of this, many studies are currently being performed at The National Institutes of Health and other research centers to determine the cause and hopefully to diminish the early onset of these adult diseases, thereby increasing the healthy life span.

This medical research has identified various factors as causes of adult disease, particularly in the middle aged and elderly population. For example, evidence suggests that insufficient blood circulation can cause serious health problems because the blood flow in the body is the means of taking the life sustaining oxygen from the lungs and critical nutrients to all parts of the body. When blood circulation is insufficient, the cells and tissues of the body do not receive their necessary life-sustaining oxygen and nutrients. When the situation persists for a period of time, organic pathological changes begin to occur in organs and tissues. Once these changes progress beyond a critical point, serious and irreversible medical conditions (e.g., heart attacks and strokes) can suddenly happen without any prior warning. For example, approximately fifty percent of individuals don't survive their first heart attack.

Current Medical Practices:

Medical research has identified certain body “conditions” that an individual might have that increases their risk of having a heart attack or stroke. These conditions include having elevated blood pressure, higher than recommended cholesterol, excess weight, and high percent body fats. Other risk conditions might include changes in electrocardiogram, distribution of the fat concentrations in the body, smoking tobacco products, and others. However, all of these preventive medicine approaches are based on statistical studies of large groups of individuals. These studies demonstrate, for example, high cholesterol increases the risk of an individual having a heart attack.

However, in reality, it is not uncommon for heart attacks or strokes to occur even in individuals who have had consistently low cholesterol values. Similarly, there are examples of elderly persons who have very high blood pressure, but still remain in good health. In fact, there are numerous instances where a substantial organic pathological change has occurred, and yet nothing has been observed in the electrocardiogram, etc.

The unfortunate fact is that the current examination equipment in most doctors' offices usually only discover substantial organic pathological changes after it has progressed to an advanced stage. Many health experts agree that this is not acceptable because the additional delay may allow irreversible and perhaps fatal damage to occur.

Thus, there is need for simple-to-administer, simple-to-understand and non-invasive tests to determine the status of a person's organic pathology. Moreover, if such non-invasive tests were available, it would allow re-measurements to be made at periodical intervals to determine the rate that organic pathological changes are occuring.

There are four different non-invasive measurements that can be made using light transmission technology at the fingertip. These measurements satisfy the need for a simple-to-administer and simple-to-understand screening test. These are: (1) Blood Circulation Analysis; (2) Heart Rate Variability; (3) Detection of irregular heartbeats; and (4) Pulse Oximetry. These four measurements are discussed briefly below.

I. Blood Circulation Analysis:

Approximately twenty-five years ago, Japanese researchers discovered a unique method of analyzing the pulse signal at the fingertip that provides a useful indicator of the quality of blood circulation. This Japanese research is summarized in several technical papers. These include: (1) Yugi Sano et al., “Evaluation of Peripheral Circulation with Accelerated Plethysmography and Its Practical Application”; J. Science of Labour, Vol. 61, No. 3, 1985; (2) “Acceleration Plethysmogram, Technical Report by the Misawa Homes Institute of Research and Development (Japan) (year unknown); (3) Katsuki K. Yomamoto et al., “A New Index of Acceleration Plethysmogram and Its Clinical Physiological Evaluation;” Nepon Seirigaku Zasshi. 1994; 56(7): 215-22; and (4) Oh-I T. Okuda et al., “An Experimental Study of Vascular Dynamics by an Acceleration Plethysmogram Using Artifical Circulation Devices,” Life Science 2002, August 23; 71(14): 1655-66.

In addition to the above technical papers, the following patents related to this field have been uncovered: (1) Japanese Patent 63-212327, A (Matsushita Electric Industrial Co., Ltd.) Sep. 5, 1988; (2) Japan Patent 2-3927, U (Nisan Motor Company, Ltd.) Jan. 11, 1990; (3) Japnese Patent 6-105829, A (Masao Sakaguchi), Apr. 19, 1994; (4) Japanese Patent 7-88092, A (Seiko Epson Corp.), Apr. 4, 1995; (5) European Patent 645117; (6) Japanese Patent 7-213499, A (Omron Corp.) Aug. 15, 1995; (7) European Patent EPO 809 965 A1, “Healthcare Device for Exercise Supporting Device.” Seiko Epson Corp, Mar. 12, 1997; and (8) U.S. Pat. No. 5,941,837 dated Aug. 24, 1999, assigned to Seiko Epson Corp., Tokyo, Japan.

The above referenced technical papers and patents describe a method of analyzing the blood pulse signal at the fingertip. Such measurement is a useful indicator of the quality of blood circulation. The technology is that blood circulation involves the heart pumping out blood, which flows through the arteries to the capillaries of the tissues and organs and then returns to the veins.

The supply of oxygen and nutrients takes place at the capillaries so that the quality of blood circulation is directly related to behavior of blood in these smallest vessels. Therefore, changes over time and the amount of blood contained in the capillaries serves as a good measure of blood circulation (namely a slight difference on arterial and venial blood pressure gives rise to differences in the nutrients supplied and in gas exchange at the capillary level). For this reason the medical researchers, as described in the above papers, believe that organic physiological changes may occur in tissues and organs if the difference in arterial blood pressure increases over a longer period of time.

One widely used method for observing the changes over time and the amount of blood contained in the capillaries is the examination of fingertip pulse waveform (the gently undulating pulse waveform obtained in the finger tip (see FIG. 1)). Unfortunately, it is difficult to interpret very small changes in these types of pulse waveforms because changes in blood circulation are small and are sensitive to changes in the organisms environment. However, these prior researchers have shown that if the Second Derivative of the pulse waveform is performed (called acceleration pulse waveform or acceleration plethysmogram) meaningful information on the status of blood circulation is obtained. FIG. 2 illustrates this acceleration pulse waveform. The figure is derived from the basic pulse waveform of FIG. 1.

This Second Derivative function provides easy to understand information. FIG. 3 provides an explanation of the various peaks and valleys of the acceleration pulse waveform. As shown in the figure, the first four peaks and valleys (identified as a, b, c and d) provide meaningful information on the quality of blood circulation.

Such analyses can be performed at various body sites such as the fingertip, ear lobe, and others that have high capillary content. However, the measurement at the fingertip is rather ideal because this is where arterial blood converts to venous blood. Also, this is the site on the body where the capillaries are most developed and the amount of blood contained in the capillaries is great. Moreover, the fingertip is ordinarily exposed.

The referenced literature and patents show that the acceleration pulse waveform can be separated into seven different “categories” as shown in FIG. 4. Category 1 is a typical shape that the acceleration waveform would have for a healthy young adult. As the person ages, or health diminishes, the relationship between point a, b, c, and d shown in FIG. 4 changes. As the category number increases, it means the person's heart is providing less of the necessary nutrients and oxygen to the tissues.

In the prior art, the fingertip pulse signal was measured using a reflectance type sensor as shown in FIG. 5 (this is FIG. 16 from U.S. Pat. No. 5,941,837, which is incorporated herein by this reference). In performing such a measurement, a light emitting diode (LED) provides illumination to the flesh portion of the most distal portion of the finger. In that same region, a reflectance silicon detector is installed. The undulating pulse signal causes pressure changes against the illumination sensor, thereby changing the reflectance reading.

Unfortunately, this reflectance signal is very small and can be easily corrupted by the method the person enters their finger into the measurement chamber. This causes a high rate of measurement rejections because either the pulse signal cannot be found or there are inconsistencies in the measurement. Moreover, none of the technical literature nor the patents describe how the basic pulse signal is then converted to Second Derivative form in order to make the measurement meaningful.

II. Heart Rate Variability:

In recent years, heart rate variability (“HRV”) has become an important diagnostic tool. A person's HRV is determined by electrocardiogram where the variability of the time between heart beats over four or five minutes is calculated either in linear or in frequency terms. In general, the more variability the heart rate (i.e., the larger the HRV), the healthier the heart. Saying this differently, when the heart is asked to respond to an outside stimulus, such as cold temperature, it is healthier to have the heart respond by having a change in heart rate.

The use of electrocardiogram for obtaining HRV has major limitations. First, it requires the use of a skilled professional to administer the test. Second, it requires the person being tested to lie down during the testing after disrobing of the chest and ankle area to allow placing of the electrodes. Third, it is an expensive test.

In the Journal of Occupational Health, 1997; 39:154-155, there was an article entitled, “Accuracy of Pulse Rate Variability Parameters Obtained from Finger Plethysmogram: A Comparison of Heart Rate Variability Parameters Obtained from ECG” (Takoyuki Kageyama, et al). This paper showed that there is a good correlation between using an ECG (sometimes called EKG) and fingertip plethysmogram using the acceleration waveform as previously described.

III. Pulse Oximetry:

Oxygen is carried in the blood attached to hemoglobin modules. Pulse Oximetry is a measure of how much oxygen the blood is actually carrying as a percentage of the maximum amount it could carry; normally called either PaO2 or PSO2. For example, a fit healthy young person will have an oxygen saturation of 95% to 99%.

Although the PaO2 can be measured at various sites on the body including ear lobe, toes or fingertips, it is most commonly measured at the fingertips. This is the same place with the same type that the previously described blood circulation analysis and heart rate variability is measured.

IV. Irregular Heart Beat:

Almost everyone has felt their heart beat very fast or felt a “fluttering” in their chest or thought their heart was “skipping a beat.” These are signs of arrhythmia or abnormal irregular heart beat and are extremely common, especially as you get older. This can occur in a healthy heart and be of minimal consequence. However, it also could indicate a serious problem due to heart disease that can lead to strokes and sudden cardiac death.

The same fingertip plethysmogram technique used for the three preceding measurements also provides a direct method of determining whether a person has an irregular heart beat.

### SUMMARY OF THE INVENTION

The present invention provides systems and methods for determining whether a subject has a physiological abnormality.

In one embodiment, the method includes the following steps: attaching a fingertip sensor to a finger of a subject, wherein, during a period of time when the fingertip sensor is attached to the subject's fingertip, the fingertip sensor outputs data; storing at least some of said outputted data; utilizing at least some of said stored data to derive a score and/or index representing a quality of the subject's blood circulation; and utilizing at least some of said stored data to: (a) determine whether the subject experienced an irregular heart beat; (b) determine the subject's heart rate variability; and/or (c) determine the subject's oxygen saturation.

In some embodiments, the fingertip sensor includes a light emitting configured to output light having a wavelength between 880 and 950 nanometers and is further configured such that it emits the light for at least a two-minute continuous period while the fingertip sensor is attached to the subject's finger. In some embodiments, during the two-minute continuous period, the fingertip sensor outputs data and at least some of the data output during the two-minute continuous period is utilized to derive the score and/or index representing the quality of the subject's blood circulation.

A system according to some embodiments of the invention includes: a fingertip sensor for attaching to a subject's finger and for outputting data that can be used to determine whether the subject has a physiological abnormality and a data collection and analysis system coupled to the fingertip sensor and configured to receive said outputted data. The data collection and analysis system includes a programmable data processor that is configured to: store at least some of said received data; utilize at least some of said stored data to derive a score and/or index representing a quality of the subject's blood circulation; and utilize at least some of said stored data to: (a) determine whether the subject experienced an irregular heart beat; (b) determine the subject's heart rate variability; and/or (c) determine the subject's oxygen saturation.

In another aspect, the invention provides a method for analyzing a subject's blood circulation. In one embodiment, the method includes: attaching a fingertip sensor to a finger of a subject, wherein, during a period of time when the fingertip sensor is attached to the subject's fingertip, the fingertip sensor outputs data; analyzing at least some of the outputted data to determine useable pulse beats; averaging the second derivative spectra of all the good pulse beats to produce an average; dividing the average by the largest value of any individual pulse beat, thereby normalizing the data; using the normalized data to derive a score and/or index concerning the subject's blood circulation. The step of analyzing at least some of the outputted data to determine useable pulse beats may comprise the step of smoothing at least some of the outputted data.

The above and other embodiments of the present invention are described below with reference to the accompanying drawings.

### BRIEF DESCRIPTION OF THE DRAWINGS

The accompanying drawings, which are incorporated herein and form part of the specification, illustrate various embodiments of the present invention. In the drawings, like reference numbers indicate identical or functionally similar elements. Additionally, the left-most digit(s) of a reference number identifies the drawing in which the reference number first appears.

FIG. 1. illustrates a conventional fingertip pulse waveform.

FIG. 2 illustrates an acceleration pulse waveform.

FIG. 3 illustrates the four points that are used for calculating a blood circulation SCORE.

FIG. 4 illustrates that that the acceleration pulse waveform can be separated into several categories.

FIG. 5 illustrates a prior art measurement system.

FIG. 6 illustrates a measurement system according to one embodiment.

FIG. 7 illustrates various components of a measurement system according to one embodiment.

FIG. 8A illustrates a fingertip pulse waveform.

FIG. 8B shows the average second derivative of the waveform shown in FIG. 8A.

FIG. 9 illustrates a common method of calculating second derivative.

FIG. 10 is an example of smoothed second derivative data.

FIG. 11 is an example of unsmoothed second derivative data.

FIG. 12 is a plot of normalized data plotted using curvilinear interpolation.

FIG. 13 illustrates the scores given to the various different shapes of the acceleration pulse waveform.

FIG. 14 is a plot of the value of SCORE versus the seven categories.

FIG. 15 serves to illustrate how a “Heart Efficiency INDEX” is developed.

FIG. 16A illustrates a typical EKG signal.

FIG. 16B illustrates the signal by using the acceleration waveform.

FIG. 17A illustrates a normal heartbeat.

FIG. 17B illustrates an irregular heartbeat.

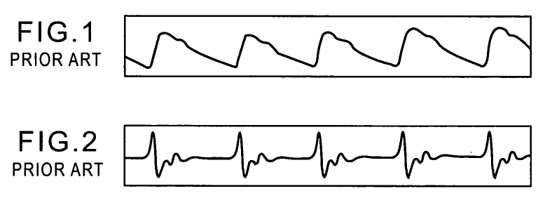

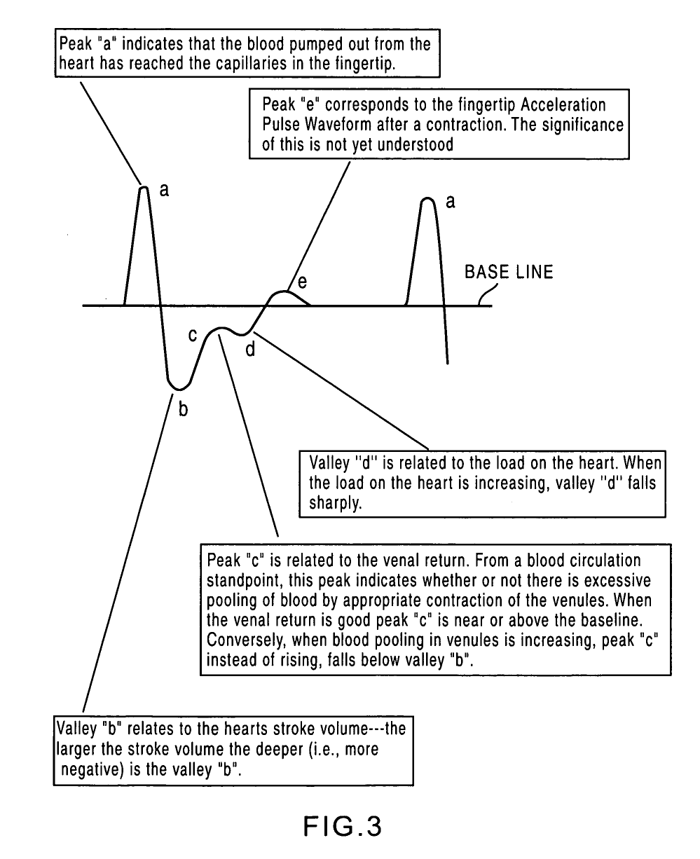

### DETAILED DESCRIPTION OF PREFERRED EMBODIMENTS

As used herein, the words “a” and “an” mean “one or more.”

As described in the Background section, each of the four measurements is well known in the art and are currently used by researchers or clinicians. However, no one has ever combined two or more of the measurements into a single measurement system to allow them to be determined essentially simultaneously. The value of such measurements is that the combination of knowledge provides greater information than any of the individual knowledge. In addition, by combining the four non-invasive measurements in a single instrument with one fingertip sensor allows them to be included in self-testing kiosks that are commonly available in pharmacies that normally only measure blood pressure and body fat.

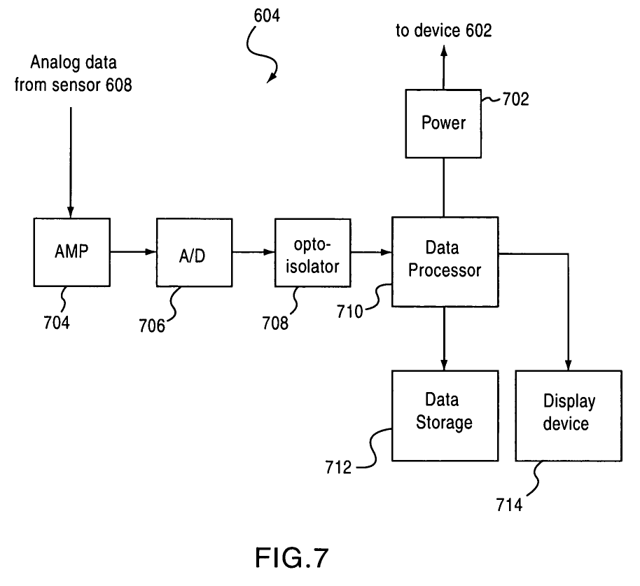

Referring to FIG. 6, FIG. 6 illustrates a measurement system 600 according to an embodiment of the invention. As illustrated, in the embodiment shown, system 600 includes a fingertip sensor 602 coupled to a data collection and analysis system 604. Data collection and analysis system provides power to and receives data from fingertip sensor 602. Fingertip sensor 602 includes one or more LEDs (sometimes called IRED depending on the emitted wavelength) 606 and one or more light sensors 608 (e.g., a photodiode). At least one of the LEDs 606 may be implemented using IRED AN304 available from Stanley Electric Co, LTD.

A commercially available fingertip sensor that can be used to implement fingertip sensor 602 is available from Dolphin Medical, Inc. (see e.g., Dolphin Model 2000), but other sensors from other companies can also be used.

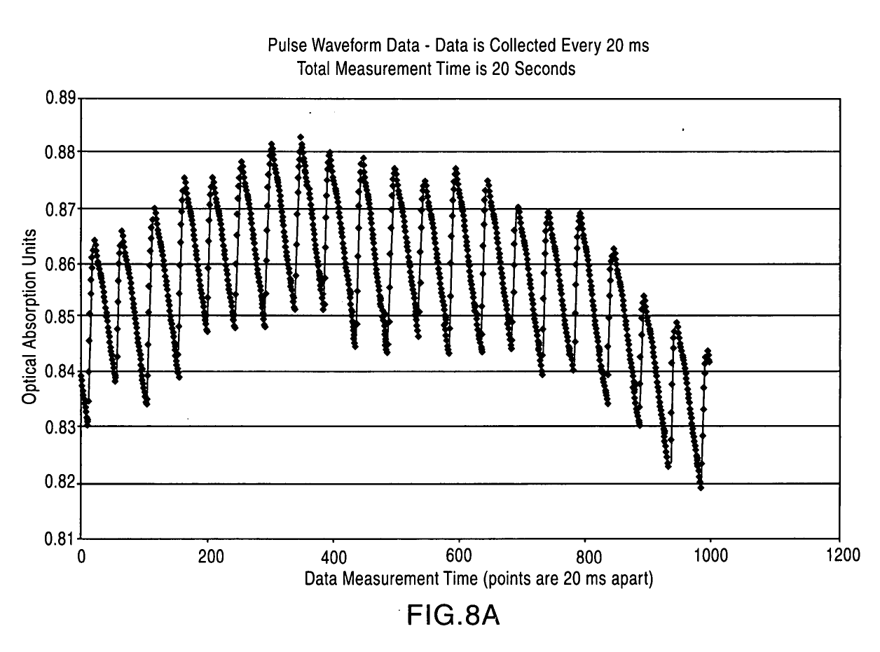

Referring now to FIG. 7, FIG. 7 is a functional block diagram illustrating various components of system 604 according to one embodiment. As illustrated, system 604 may include: (a) a power source 702 for providing power to fingertip sensor 602; (b) an amplifier 704 for receiving and amplifying analog data output from sensor 602 (preferably, the amplifier is a low noise amplifier); (c) an analog-to-digital (A/D) converter 706 for converting the output of the amplifier into digital data (preferably, A/D converter is a precision high speed, high resolution A/D converter); and (d) a data processor 710 for receiving, storing and processing the digital data.

In some embodiments it may be desired to electrically isolate the data processor 710 from the A/D converter 706. In such embodiments, an optoisolator may be used to couple A/D converter 706 to data processor 710.

Data processor 710 may be a general purpose computer programmed to (I) perform blood circulation analysis for a test subject, (II) measure the test subject's hear rate variability, (III) measure the test subject's oxygen saturation, and (IV) detect whether the test subject has an irregular heart beat.

I. Blood Circulation Analysis

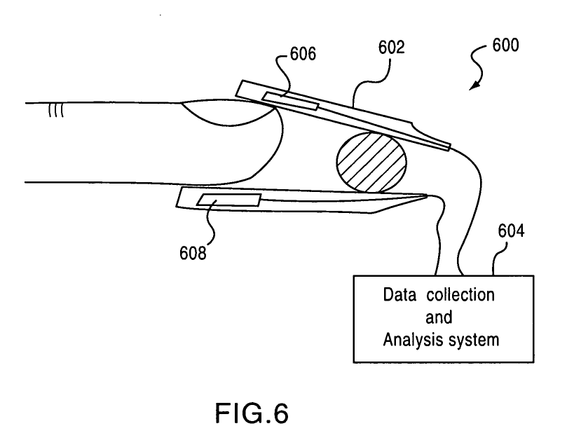

As shown in FIG. 5, the prior art used reflectance measurement off the pad of the most distal portion of the finger to perform the measurement. But, the inventor recognized that considerably more measurement signal, thus providing a more reliable measurement, can be achieved by performing a light transmission measurement through the most distal portion of the finger (see FIG. 6). Such measurement, similar to the measurement that is widely used in pulse oximetry, shows consistent measurement of the pulsal waveform.

The recommended embodiment is to use the transmission measurement at a single wavelength, preferably between 880 and 950 nanometers. By having the LED 606 stay on continuously, precise measurement of the waveform can be achieved. Studies have shown that data should be collected at least every 20 milliseconds in order to properly define the pulse waveform. Moreover, such measurements should be made over a number of pulse beats and then averaged in order to reduce noise and provide the average pulse waveforms. Accordingly, data processor 710 may be programmed to collect a data point every 20 milliseconds, where each data point corresponds to the light measured by sensor 608 at a particular point in time. Accordingly a data point may be associated with two values: (1) a value representing optical absorption and (2) a time or sequence value (e.g., a scan number). The collected data point may be stored in storage device 712.

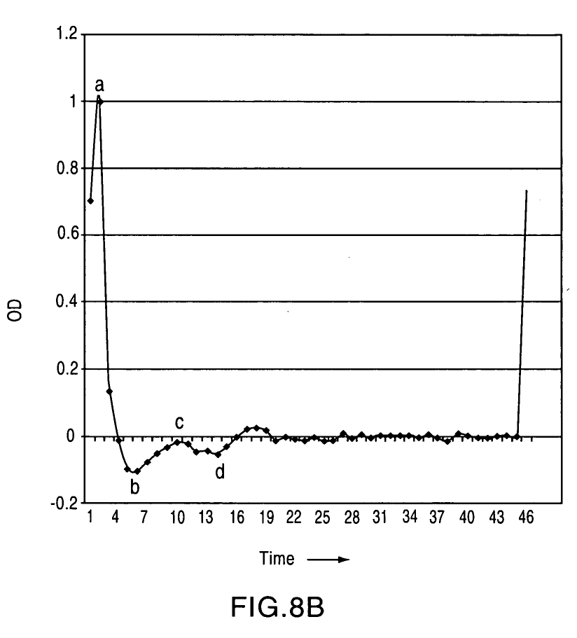

FIG. 8A is a typical waveform from a woman based on performing measurements every 20 milliseconds for a period of twenty seconds. Twenty seconds of measurement provides twenty pulse waves for someone whose pulse rate is 60 beats per minute (approximately the average of adults). For those with a higher pulse rate more averages is available. For those with slow pulse rates, somewhat less data is available, but still sufficient to allow precise measurement.

FIG. 8B shows the average second derivative (the “Acceleration Pulse Wave”) for the same individual.

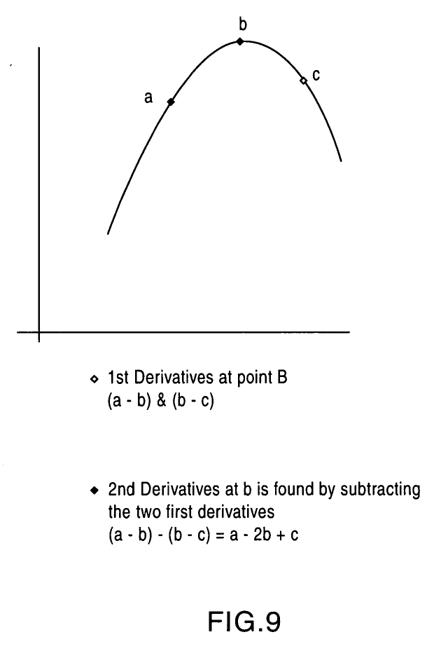

As illustrated in FIG. 9, a common method of calculating second derivative is by subtracting the slopes on each side of the desired point. Using this approach, the second derivative is equal to [a−2b+c]. The questions arise on (i) how far should a be apart from b and (ii) how far should b be apart from c (the smaller the distance, the more meaningful information.) However, a small distance could introduces undesirable noise in the second derivative calculation. But, because small inflections in the pulse cyclic data contains important information, the smaller gap provides the best opportunity to capture that signal. If the noise level is high enough to hide the signal the gap between a to b and b to c must be increased. The larger gap does reduce the noise but introduces undesirable artifacts that may eliminate the sensitivity to small important inflections in the raw data.

An alternate approach is to use the Savitsky-Golay approach. This approach provides a good signal to noise but is somewhat complex mathematically. In the preferred embodiment, the calculation may be performed as described below.

TASK “A”—Obtaining Essentially Noise Free Optical Data.

Because of the sensitivity of noise interfering with meaningful second derivative data, preventing noise from interfering with the measurement may be desired, but is not required. Lowering the impact of noise may be accomplished by: (1) using fingertip sensor 602 in the transmission mode with an LED left on in a continuous basis (in this approach much higher resolution than the conventional reflectance approach is obtained); (2) utilizing a precision high-speed A/D converter to obtain 128 individual measurements during the 20 ms period and obtaining the average, thereby minimizing the random noise; (3) using a low noise electrical amplification system to obtain the measured pulsal signature at every 20 ms; (4) using an A/D converter that provides a high resolution (e.g., nineteen bits of resolution); (5) operating the fingertip sensor 602 directly off of batteries as opposed to an AC adapter; and (6) decreasing the possibility of the person's body acting as a receiving antenna from random electrical noise due to radio stations and TV signals (this may be accomplished by grounding the person to the same ground as the analog portion of the measurement system 600).

TASK “B”—Locating Every True Pulse Beat:

In order for the analysis to be performed, care must be taken to insure that only true pulse beats are recorded at the proper intervals. We had discovered that an excellent means of determining true pulse beats is to smooth the collected data point by averaging the ODs of five adjacent OD terms. Saying this differently, the OD at any instant is the value of the OD measured at that instant plus the value of the two OD terms just prior to that instant plus the two OD terms just following that instant. This total is then divided by five to provide the OD term at that instant.

Previous research has shown that using a second derivative with gap=±1 of the data file when the OD term has been smoothed by the averaging 5 OD of five adjacent OD terms provides an excellent means of determining the pulse peak. The method used is defined in the following three steps.

Step 1—Converting A/D data to OD

The “OD” is defined by the following Equation: OD=Log(S/(AD/gain)), where “S” is the full scale value of the linear measuring analog to digital converter; “AD” is the value of the linear signal being measured; and “Gain” is the sensitivity of the measurement system (typically 1, 10 or 100).

Step 2—Smoothing the A/D Data

The OD resulting from Step 1 is to be smoothed by calculating the running average so that each data point would be the average of five scan numbers.
OD sm₅=(ODᵢ₋₂+ODᵢ₋₁+ODᵢ+ODᵢ₊₁+ODᵢ₊₂)/5.

Step 3—Calculating the Second Derivative with Gap=±1

Second derivative at any scan #=ODᵢ₋₁−2ODᵢ+ODᵢ₊₁

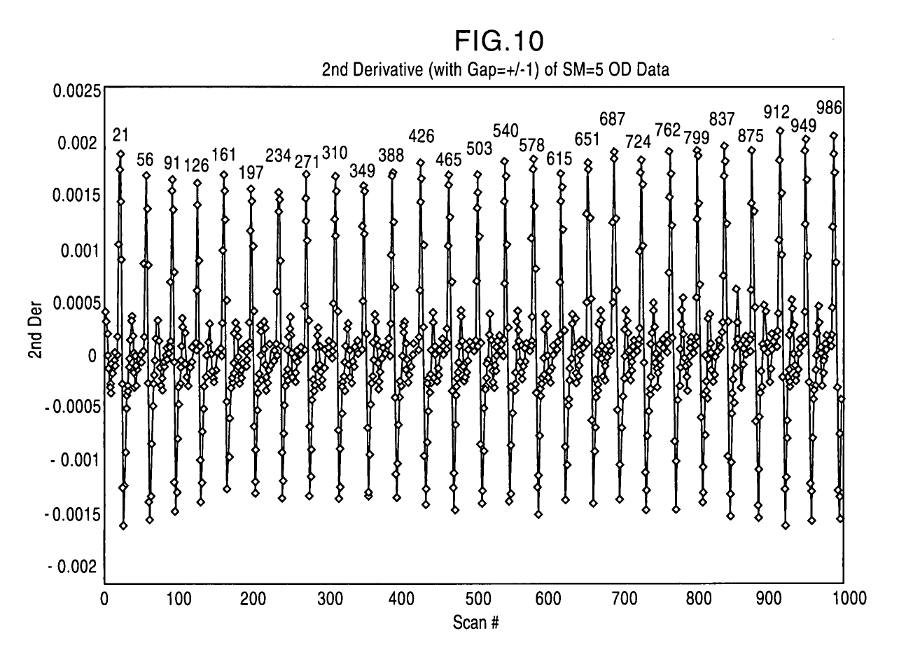

FIG. 10 is an example of the second derivative data for smoothing=5. Written onto FIG. 10 are the scan numbers of every pulse peak.

You will note that the smallest peak occurs at Scan #234 which has a second derivative value of approximately 0.0016. Moreover, the largest noise between pulse peaks is shown approximately midway between Scan #837 and #875. It has a second derivative value of approximately 0.0006. It is believed that a reasonable tolerance for allowable maximum noise would be approximately 60% of the minimum true second derivative pulse point. For FIG. 10 this means the 0.6×0.0016=0.001. Thus, the maximum noise in this example (i.e., 0.0006) is well within the allowable tolerance, thereby assuring that the selection of actual pulse points is realistic.

TASK C—Doing Analysis Without Smoothing

As previously stated, smoothing tends to “wash out” subtle differences that may occur. For the blood circulation analysis it is desired to actually measure these subtle differences. Thus, the data analysis must be done for no smoothing.

Calculating Second Derivative without Smoothing:

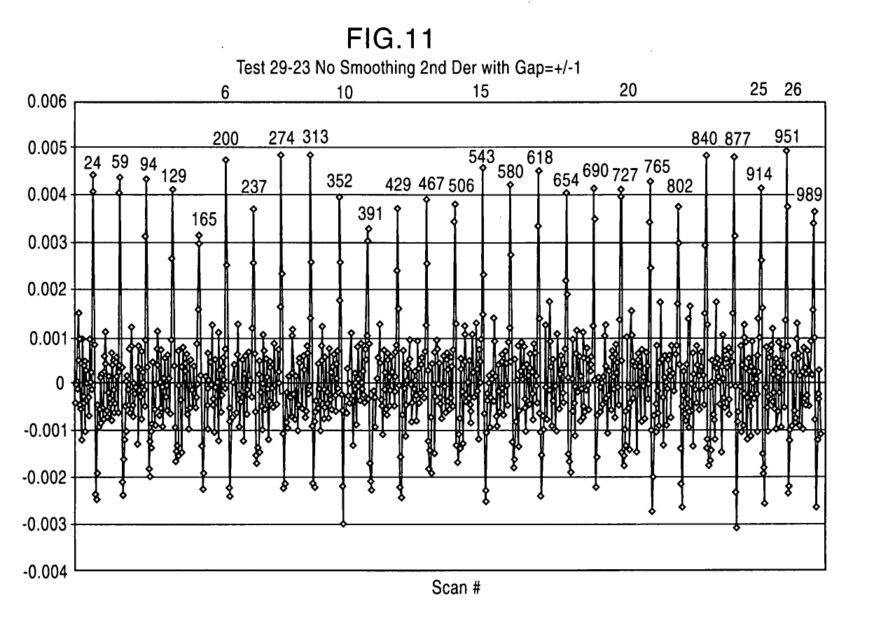

The second derivative should be calculated using the same equation as was used in Step 3, however, it should be performed on the unsmoothed OD data. Shown on FIG. 11 are the scan numbers of the peak positive values of the second derivative curve without any data smoothing.

Comparing the scan numbers of FIG. 11 with FIG. 10, illustrates that the unsmoothed data has positive peaks approximately three scan numbers larger than the smoothing=5 data. Thus, although the smoothing=5 data determines the existence of the pulse peak, the data from FIG. 11 (the unsmoothed data) defines the true scan number that is to be used in the subsequent analysis.

TASK D—Defining Missing or Non Usable Pulse Beats

Approximately one out of ten normal people occasionally skip a heart beat. The required blood circulation analysis must not let such a skipped heart beat corrupt the results. A second possible cause of corruption is if there is a large noise spike, for example, by the person accidentally hitting the fingertip sensor against a hard surface. The following analysis is used to locate the missing pulse beat or false pulse beats.

Determining Usable Pulse Beats:

First, compare the second derivative curve obtained from the unsmoothed OD data to the second derivative curve obtained from the smoothed OD data to find those pulse beats from the unsmoothed data whose scan number is not within three scan numbers of any scan number corresponding to a pulse beat from the smoothed data. Those determined pulse beats are not useable and are eliminated.

Next, the number of scans between pulse beats should be determined for every remaining pulse beat from the unsmoothed data. For example, the number of scans between the first and the second pulse beat in that figure is 59−24=35. Similarly, the number of scans between the next pulse beat would be 94−59=35. The largest number of scans between any two adjacent pulse beats occurs between #391 and #352, a difference of 39.

The difference in scan numbers between every adjacent pulse beat needs to be determined. Then the average of these differences is calculated. For the example shown in FIG. 11, the average number of scans between pulse beats is 37.12 scans. Acceptable pulse beats are those in which the number of scans between it and the adjacent pulse beat is within ±15% of the average value.

If any pulse beat is found to violate this acceptance criteria, it should be eliminated and the average recalculated. On the recalculated average, the difference between pulse beats, except for the location where the pulse beat had been eliminated, should be within ±15% of the average.

The results of this analysis provides the meaningful pulse beats that exist.

TASK E—Determining the Shape of the Average Second Derivative for Good Pulse Beats

In order to minimize noise, the second derivative spectra of all the good pulse beats must be averaged. This section describes how to perform this task (Note, in the following steps the highest scan number peak should not be included—for example, in FIG. 11 Scan #989 should not be used).

Adding all Scans Together:

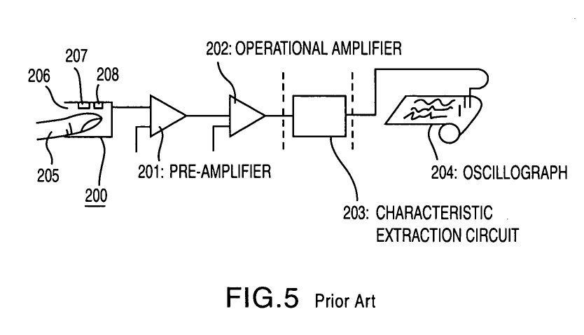

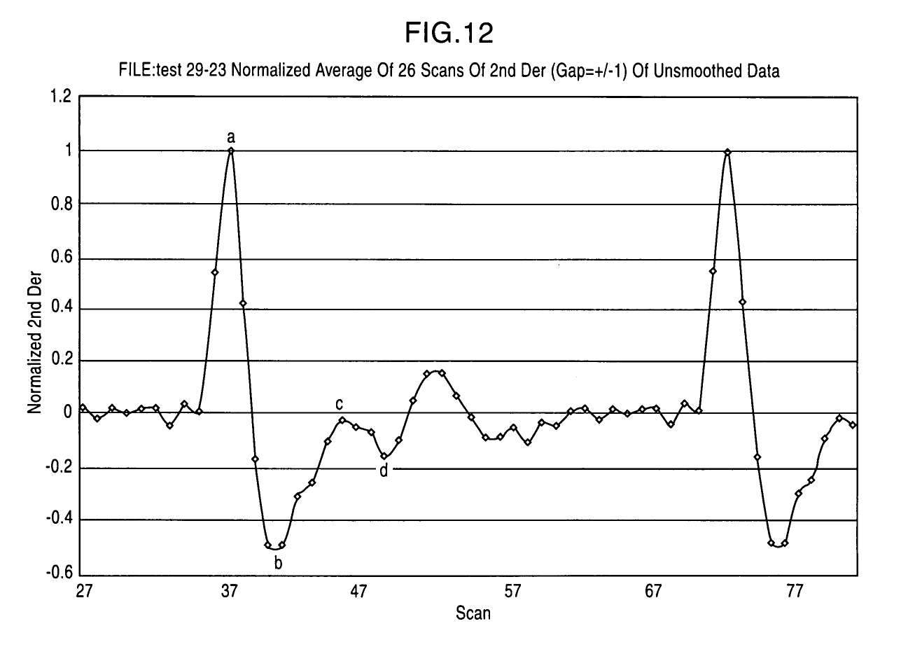

All usable pulses from the second derivative of the unsmoothed data are then averaged. The average value is then divided by the largest value of any individual pulse beat (i.e., the maximum positive value), thereby normalizing the data. FIG. 12 is a plot of the normalized data plotted using curvilinear interpolation. It then is available to be compared to the seven blood circulation categories defined in FIG. 4.

Calculation of “SCORE”:

The prior art defined a quantitative numerical measurement parameter given the name of “SCORE.”

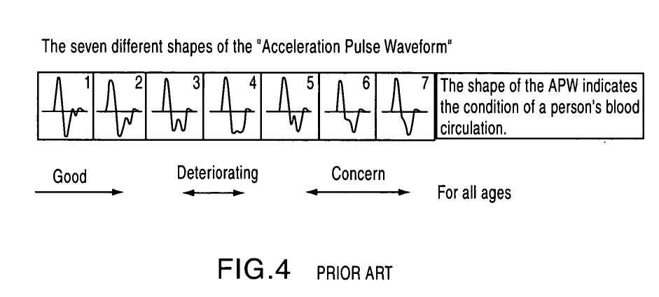

In FIG. 3, the four points that are used for calculating the SCORE are shown; points a, b, c and d. The calculation is as follows: SCORE=100(−b+c+d)/a

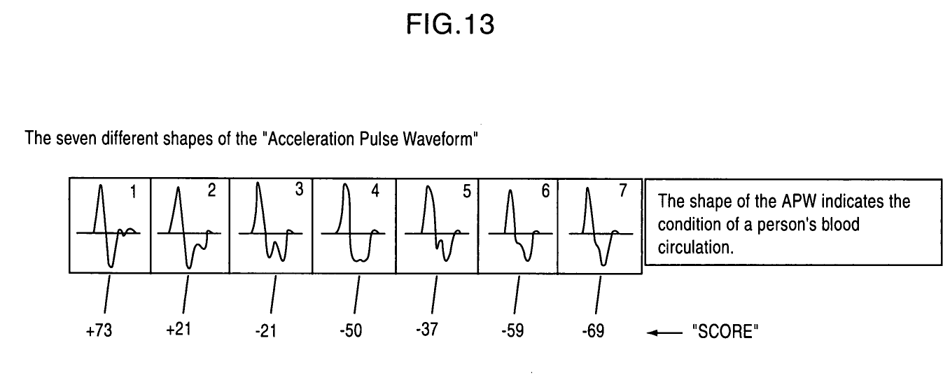

As shown in FIG. 13, the value of SCORE varies from +73 for Category 1 to a −69 for Category 7. The drawbacks of such a “plus to minus” type of scale is that the lay person finds it difficult to understand the value of the particular SCORE from their test. What is needed is a simpler linear scale in order to provide the person being tested with a meaningful measure of what that person's rating is.

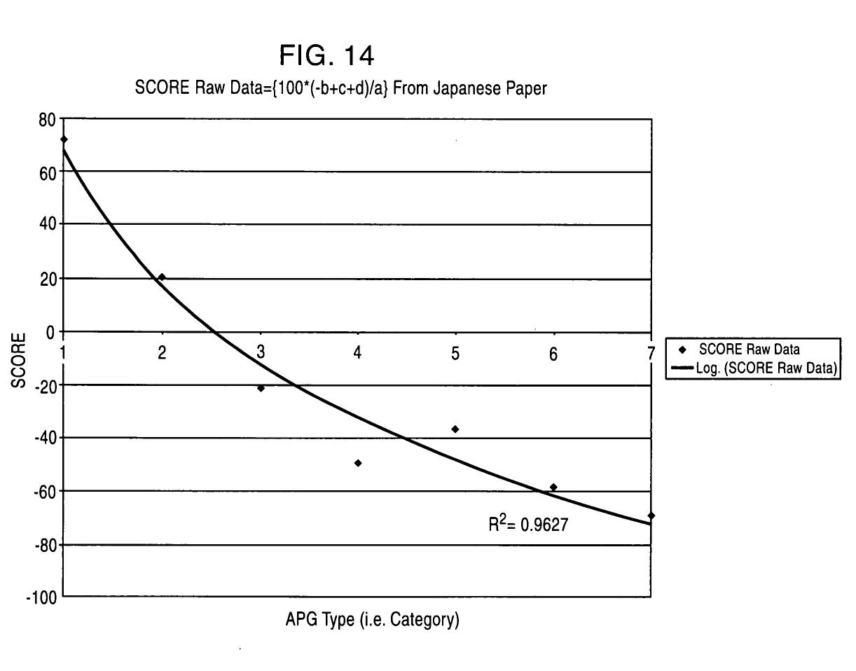

To derive such a meaningful rating system, the value of SCORE versus the seven categories was cross plotted in FIG. 14. As shown in the figure, a logarithmic regression line provides an R² of 0.9627. This very high R² indicates that a meaningful linear rating system can be developed.

To develop a meaningful “Heart Efficiency Index” it was decided to define a Category 1 healthy heart to have a INDEX of 90%, and a heart in Category 7 would have an INDEX of 20%. Although these two limits are somewhat arbitrary, it does allow a meaningful index to be derived.

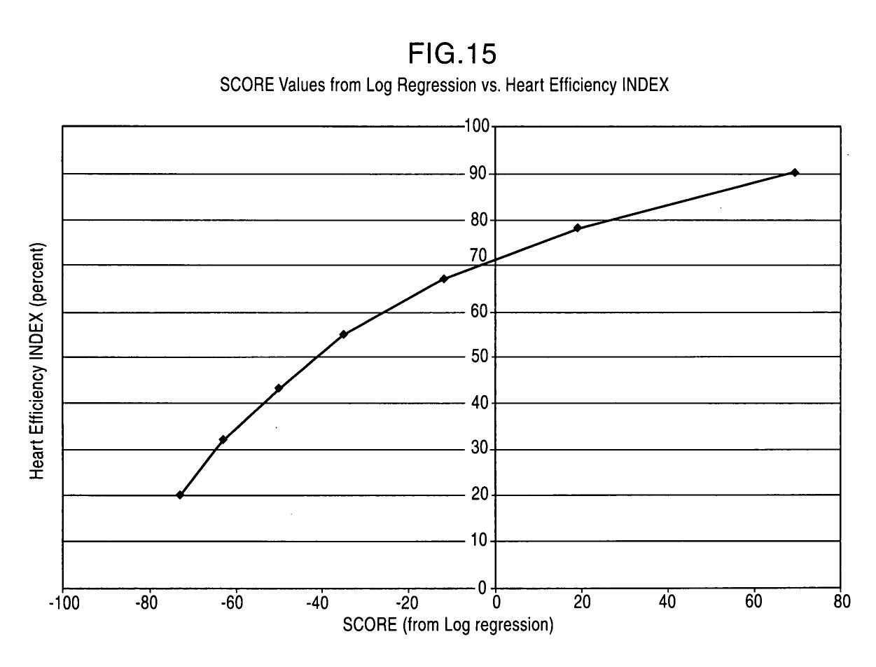

FIG. 15 shows how the “Heart Efficiency INDEX” was developed. To study the value of this INDEX, a group of twenty-four individuals chosen at random had their blood circulation analyzed by the definitions contained in this Disclosure. They were each given both their SCORE value and their Heart Efficiency INDEX values. They were then given a questionnaire concerning which of the two results had the most meaning to them. Of the twenty-four volunteers, twenty-three stated that the Heart Efficiency INDEX was much easier to understand compared to the SCORE concept. The other person said that they had no preference of one versus the other.

II. Heart Rate Variability

The traditional method of determining heart rate variability is to use an electrocardiogram. Such testing requires disrobing and, in general, and normally to have to have the person lie down on one's back. Such an approach is suitable for a medical office. However, it is not suitable for a population screening test in order to find people that have unknown medical problems.

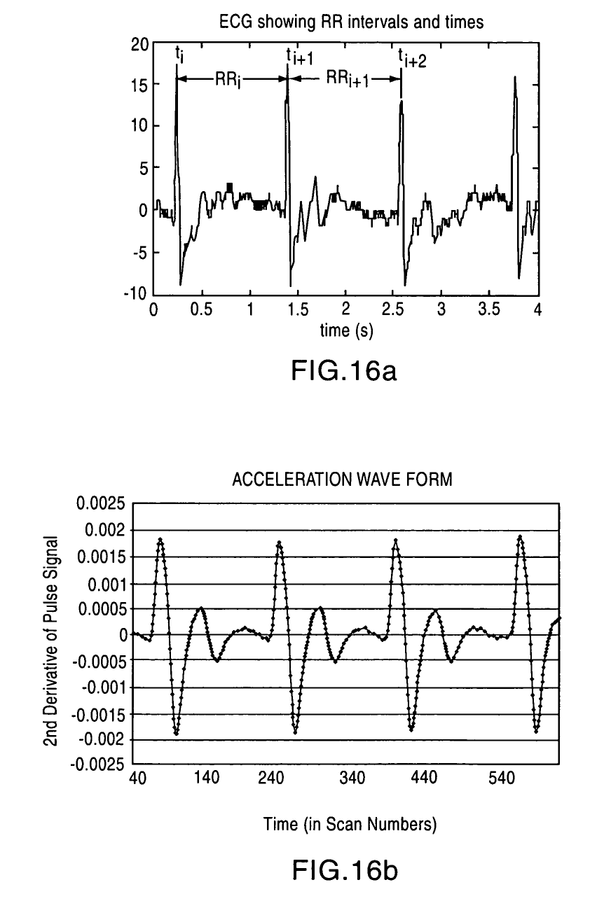

FIG. 16a illustrates a typical EKG signal. FIG. 16b illustrates the signal by using the acceleration waveform as described in the blood circulation analysis. The similarity between the two figures is obvious.

Moreover, as described in the referenced article (Journal of Occupational Health, 1997; 39:154-155) there is a high correlation in determining HRV using the EKG method compared to using acceleration waveform analysis. In addition to this fact, is the knowledge that many, perhaps a majority, of commercial pharmacies have kiosks that currently measure blood pressure. Such measurements are normally made while the person is seated and their arm is through some type of fixed located cuff. During the cycle of such measurement, if a fingertip sensor is used, the measurement of HRV over a fixed period of time can also be determined.

Traditional HRV testing is for a minimum of four or five minutes. However, reasonable HRV data can be determined during screening tests while the person is in the seated position over a two-minute period. This means that by using fingertip sensor 602, the measurement of blood circulation analysis, heart rate variability analysis, pulse oximetry analysis, and regular heart beat can all be performed near simultaneously.

Once the measurement is made, the mathematics to reduce the measured data to HRV standard format, either in “time domain” (Example: Calculation of the Standard Deviation of the beat-to-beat time intervals), or in “frequency domain” (Example: Use of discrete Fourier time series to the beat-to-beat interval).

III. Pulse Oximetry

While PaO2 can be measured at various sites on the body including ear lobe, toes or fingertips, it is most commonly measured at the fingertips using a fingertip sensor. Because a fingertip sensor is used to perform the blood circulation analysis and heart rate variability analysis, the same fingertip sensor can be configured to measure pulse oximetry before or after the blood circulation and heart rate variability analysis. Accordingly, the measurement system may be programmed to measure the subject's oxygen saturation before or after the blood circulation analysis. Using a finger-clip sensor to measure pulse oximetry is well known in the art (see e.g., U.S. Pat. No. 5,490,523). Thus, for the sake of brevity, the details are omitted.

IV. Irregular Heart Beat

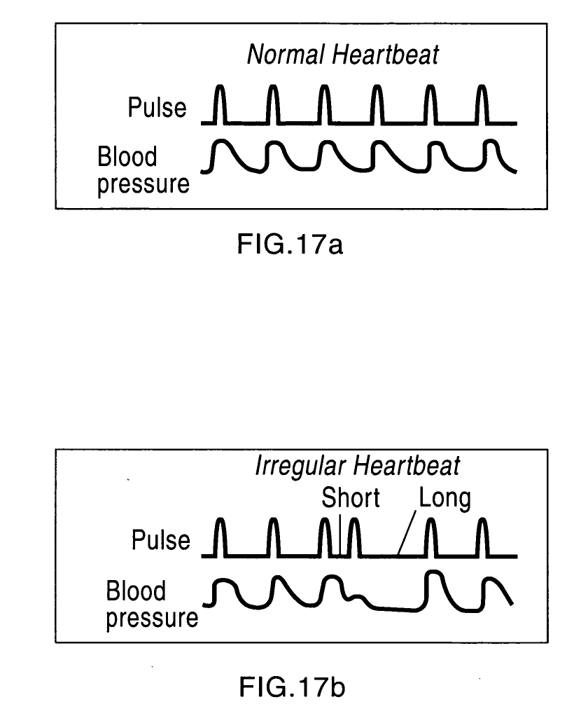

As previously described, the presence of irregular heart beat may be an indicator of serious medical problem. During the BCA measurement and/or during the HRV measurement and/or during the pulse oximetry measurement, the time between adjacent heart beats may be determined by the measurement system. FIG. 17 is a typical definition of how an irregular heartbeat is identified. In this figure, when the time between a heart beat and a previous heartbeat differs by more than 25% of the average time between heart beats the heart beat is defined as an irregular heartbeat and should be brought to the attention of the test subject. Accordingly, the measurement system may be programmed to determine the time between a heart beat and a previous heart beat and compare the determined time to a predetermined value to determine whether the heartbeat is irregular. If the system determines that the heartbeat is irregular, the system may notify the test subject that an irregular heartbeat was detected by, for example, displaying a message on a display screen of the measurement system.

### CONCLUSION

The ability of providing two or more non-invasive health screening measurements using the disclosed measurement system 600 allows a unique multi-use instrument to be offered to the medical community. Additionally, the system may be an ideal addition to commercial kiosks to allow self-testing, thereby enabling a subject to obtain knowledge about the subject's health. This knowledge allows the subject when visiting a doctor to provide information to the doctor to assist the doctor in a diagnosis.

While various embodiments/variations of the present invention have been described above, it should be understood that they have been presented by way of example only, and not limitation. Thus, the breadth and scope of the present invention should not be limited by any of the above-described exemplary embodiments.

What is claimed is:

1. A method for detecting whether a subject has a physiological abnormality, comprising:
attaching a fingertip sensor to a finger of a subject, wherein, during a period of time when the fingertip sensor is attached to the subject's fingertip, the fingertip sensor outputs data;
storing at least some of said outputted data;
utilizing at least some of said stored data to derive a score and/or index representing a quality of the subject's blood circulation; and
utilizing at least some of said stored data to: (a) determine whether the subject experienced an irregular heart beat; (b) determine the subject's heart rate variability; and/or (c) determine the subject's oxygen saturation.

2. The method of claim 1, further comprising: utilizing at least some of said stored data to (a) determine whether the subject experienced an irregular heart beat; (b) determine the subject's heart rate variability; and (c) determine the subject's oxygen saturation.

3. The method of claim 1, further comprising: utilizing at least some of said stored data to determine whether the subject experienced an irregular heart beat.

4. The method of claim 1, further comprising: utilizing at least some of said stored data to determine the subject's heart rate variability;

5. The method of claim 1, further comprising: utilizing at least some of said stored data to determine the subject's oxygen saturation.

6. The method of claim 1, wherein the fingertip sensor comprises a light emitting diode and a photodiode.

7. The method of claim 6, wherein the light emitting diode is configured to output light having a wavelength between 880 and 950 nanometers.

8. The method of claim 7, wherein the fingertip sensor includes a second light emitting diode, which light emitting diode is configured to output light having a wavelength less than 880 nanometers.

9. The method of claim 7, wherein the light emitting diode is configured such that it emits said light for at least a two-minute continuous period while the fingertip sensor is attached to the subject's finger.

10. The method of claim 9, wherein, during said two-minute continuous period, said fingertip sensor outputs data and at least some of said data output during said two-minute continuous period is utilized to derive said score and/or index representing the quality of the subject's blood circulation.

11. The method of claim 10, wherein at least some of said data output during said two-minute continuous period is utilized to determine whether the subject experienced an irregular heart beat and/or determine the subject's heart rate variability.

12. The method of claim 10, wherein at least some of said data output during said two-minute continuous period is utilized to determine whether the subject experienced an irregular heart beat and is utilized to determine the subject's heart rate variability.

13. The method of claim 10, wherein at least some of said data output during said two-minute continuous period is utilized to determine the subject's heart rate variability.

14. A system for detecting whether a subject has a physiological abnormality, comprising:
a fingertip sensor for attaching to a subject's finger and for outputting data that can be used to determine whether the subject has a physiological abnormality;
a data collection and analysis system coupled to the fingertip sensor and configured to receive said outputted data, wherein the data collection and analysis system comprises a programmable data processor, wherein the programmable data processor is configured to:
store at least some of said received data;
utilize at least some of said stored data to derive a score and/or index representing a quality of the subject's blood circulation; and
utilize at least some of said stored data to: (a) determine whether the subject experienced an irregular heart beat; (b) determine the subject's heart rate variability; and/or (c) determine the subject's oxygen saturation.

15. The system of claim 14, wherein the data collection and analysis system further comprises:
an amplifier configured to amplify said outputted data; and
an analog-to-digital converter coupled to the amplifier and coupled to the programmable data processor and configured to convert said amplified data to digital data so that said outputted data can be processed by said programmable data processor.

16. The system of claim 15, further comprising an optoisolator coupled between the analog-to-digital converter and the programmable data processor.

17. The system of claim 14, wherein the fingertip sensor comprises a light emitting diode and a light sensor.

18. The system of claim 17, wherein the light emitting diode is configured to output light having a wavelength between 880 and 950 nanometers.

19. The system of claim 18, wherein the light emitting diode is configured such that it emits said light for at least a two-minute continuous period while the fingertip sensor is attached to the subject's finger.

20. The system of claim 17, wherein the fingertip sensor includes a second light emitting diode, which light emitting diode is configured to output light having a wavelength less than 880 nanometers.

21. The system of claim 14, wherein the programmable data processor is configured to utilize at least some of said stored data to: (a) determine whether the subject experienced an irregular heart beat; (b) determine the subject's heart rate variability; and (c) determine the subject's oxygen saturation.

22. The system of claim 14, wherein the programmable data processor is configured to utilize at least some of said stored data to determine whether the subject experienced an irregular heart beat.

23. The system of claim 14, wherein the programmable data processor is configured to utilize at least some of said stored data to determine the subject's heart rate variability.

24. The system of claim 14, wherein the programmable data processor is configured to utilize at least some of said stored data to determine the subject's oxygen saturation.

25. A method for analyzing a subject's blood circulation, comprising:
attaching a fingertip sensor to a finger of a subject, wherein, during a period of time when the fingertip sensor is attached to the subject's fingertip, the fingertip sensor outputs data;
analyzing at least some of the outputted data to determine useable pulse beats;
averaging the second derivative spectra of all the good pulse beats to produce an average;
dividing the average by the largest value of any individual pulse beat, thereby normalizing the data;
using the normalized data to derive a score and/or index concerning the subject's blood circulation.

26. The method of claim 25, wherein the step of analyzing at least some of the outputted data to determine useable pulse beats comprises the step of smoothing at least some of the outputted data.
